(19) United States
(12) Patent Application Publication
(10) Pub. No.: US 2018/0185013 A1
(43) Pub. Date: Jul. 5, 2018

(54) Tidy specimen

(71) Applicant: Cynthia K. Bolden-Gardner, Canton, MS (US)

(72) Inventor: Cynthia K. Gardner, Canton, MS (US)

(21) Appl. No.: 15/732,715

(22) Filed: Dec. 19, 2017

Related U.S. Application Data
(60) Provisional application No. 61/965,554, filed on Feb. 3, 2014.

(51) Int. Cl. A61B 10/00; A61F 5/455; A61F 5/453

(52) U.S. Cl. CPC A61B 10/007; A61F 5/453; A61F 5/4556

(57) ABSTRACT

A plastic injection molded funnel for the passage of substances, including but not limited to, urine for hygiene and sanitation purposes. The funnel may be used with a locking basecap, septa flexible cap or with a locking extension containing four descending consecutively smaller cylinders for indoor and outdoor use, respectively. A flexible plastic caddy attachable to any commode may be utilized with or without the funnel and standardized medical urine specimen cup.

TIDY SPECIMEN: (the cup is NOT part of the application) This invention consists of a plastic injection molded ‘funnel’ with a spout on the front and rear end, a detachable cap for the base. This base is for use in specimen taking to secure the contents of the funnel and allow the same to be safely poured into a standard specimen cup.

There are also four locking descending cylinders, which are each detachable. These cylinders replace the cap and are for use other than medical office specimen taking.

The dimensions of the funnel are approximately 7-8″ oblong, rectangular like a boat, 2″ across the middle and 5-6″ in depth with an additional 2-3″ consisting of 4 interlocking cylinders.

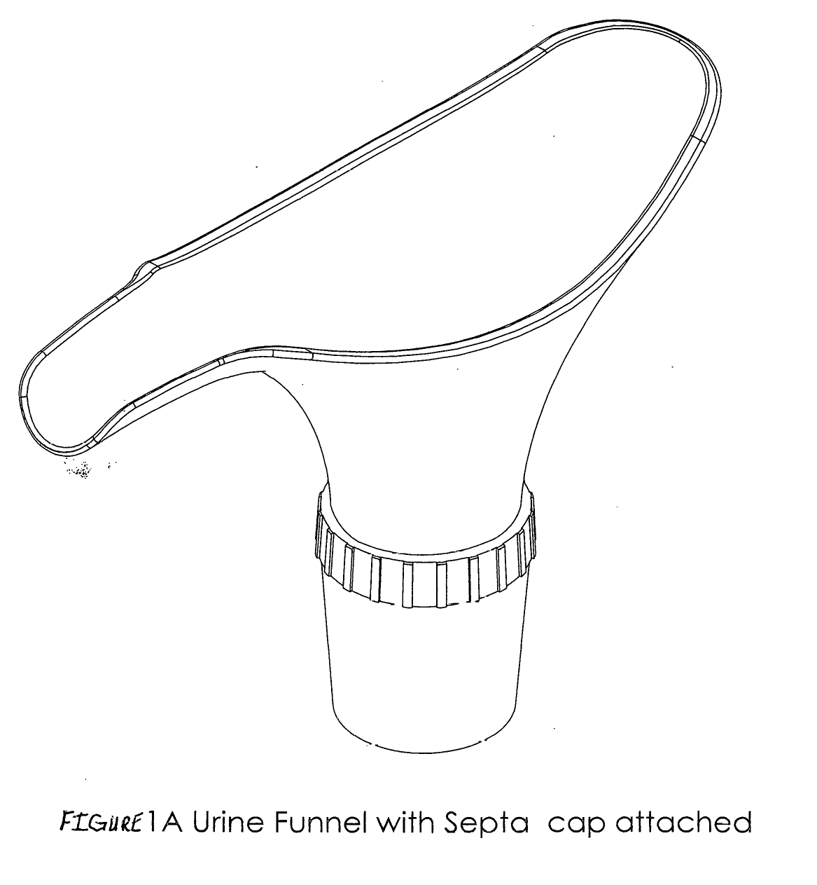

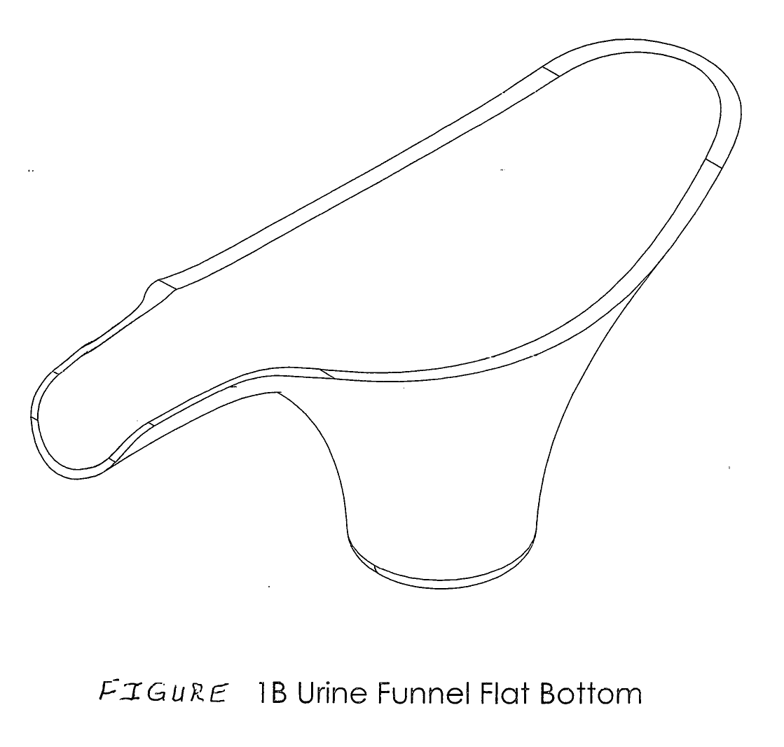

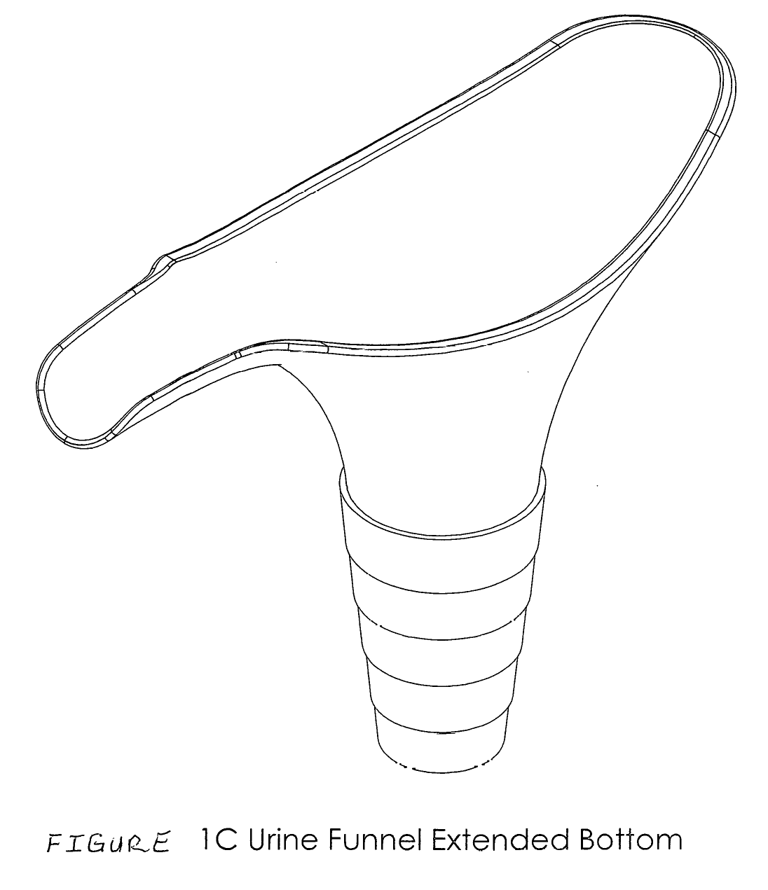

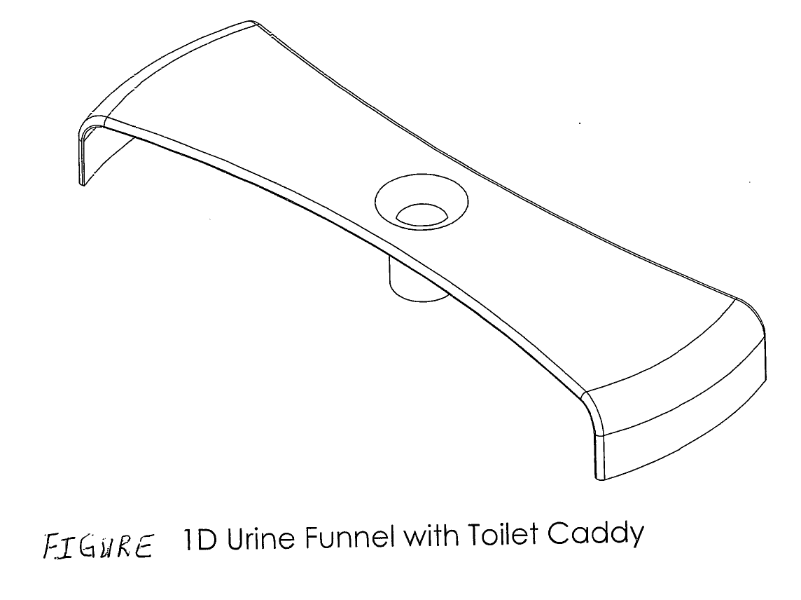

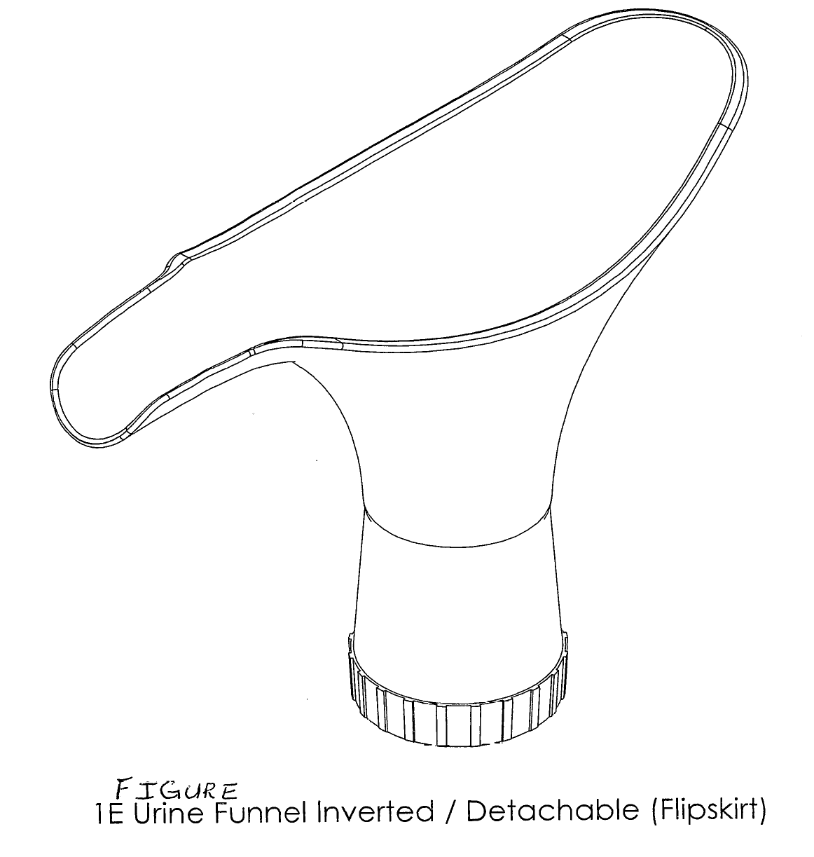

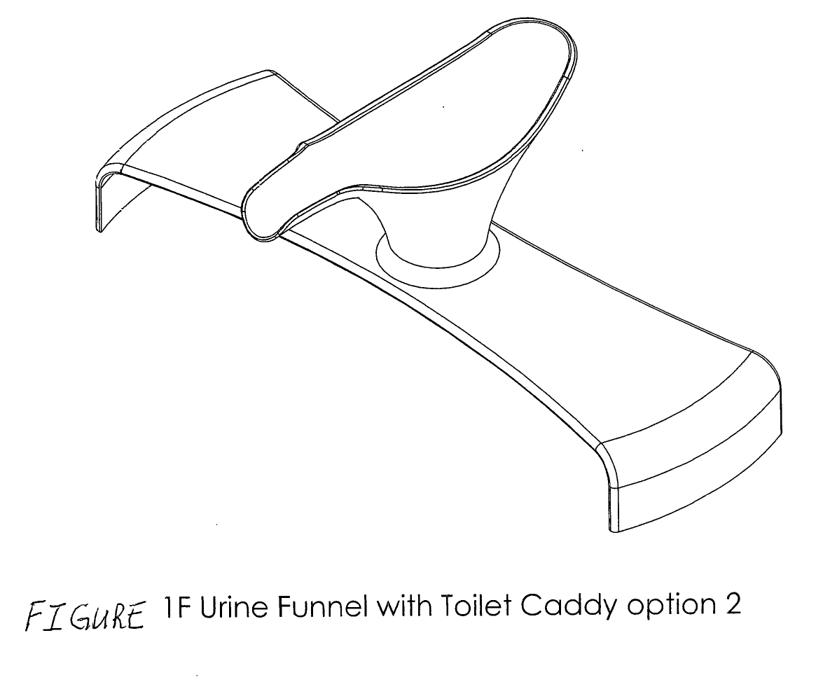

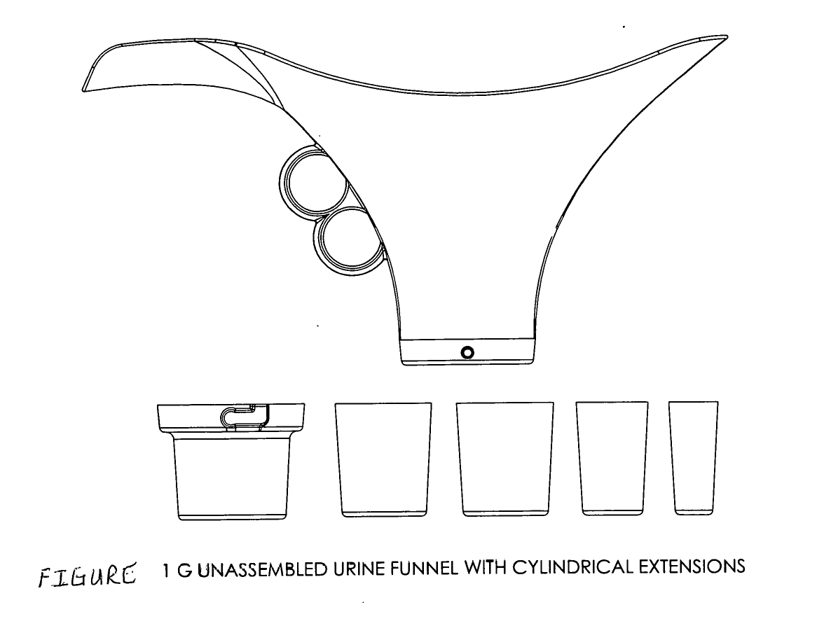

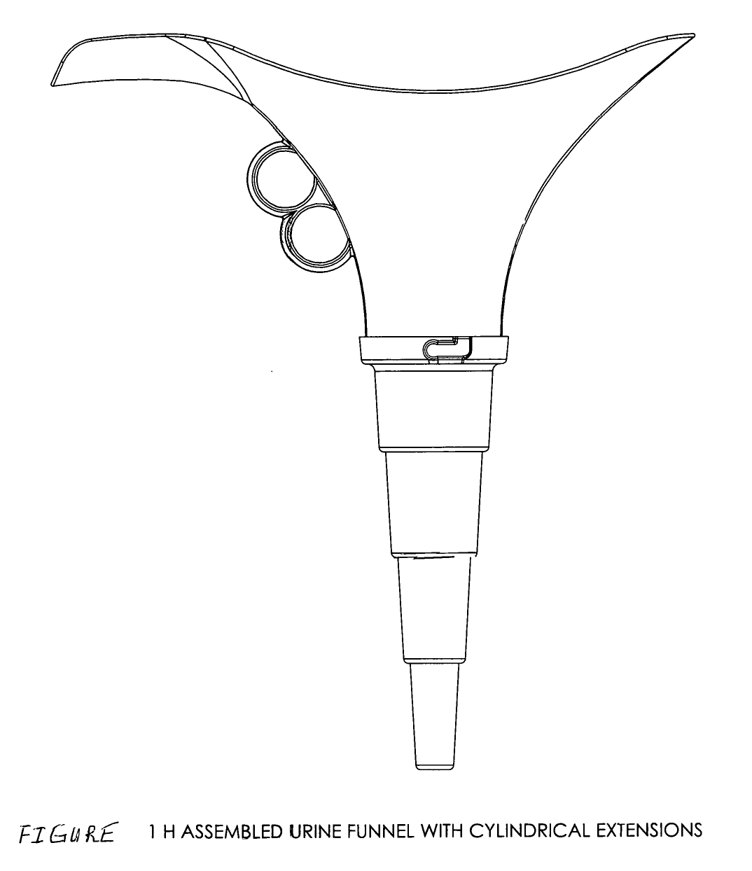

### DRAWING FIG. 1A URINE FUNNEL WITH SEPTA CAP ATTACHED

This is a right side angle view. The design is to allow the use of the funnel for medical purposes. The Septa cap is consistent with the attachment for nursing mother's breast pumps. The cap attaches the standard urine specimen cup to the funnel. The cap is rubberized to allow it to stretch to fit any specimen cup size. Recently, it appears the medical industry has resorted to using various size cups. The septa cap will accomodate those variations.

DRAWING FIG. 1B URINE FUNNEL FLAT BOTTOM

This depiction is the same as that of the urine funnel in Drawing FIG. 1A, with a right side angle view, except that it has a standard flat bottom. It is just the funnel from the same right side angle view with the capped off bottom. The cap is actually a locking cap as depicted by the small rim at the bottom and the line in the center rear.

This locking cap can be seen in Drawings FIG. 1 G-H. It is less than ½ inch in depth and twists onto the bottom or base of the funnel. As indicated in the previously filed application, it can be used for medical specimens, liquids or powders, beach use as a toy and varied other recreational and culinary uses.

DRAWING FIG. 1C URINE FUNNEL EXTENDED BOTTOM

This is the identical funnel depicted from the right angle in drawings FIGS. 1A and 1C except the flat based cap has been replaced with a locking cap with three descending cylinders that securely fit inside of each other. The purpose of the descending cylinders is primarily for unsanitary restroom facility, porta pottie, outdoor sports and recreational use. However, as heretofore stated, it can be acquired for use as a toy to build fortress prongs on a sand castle, sift culinary liquids and powders and the like.

DRAWING FIG. 1D URINE FUNNEL WITH TOILET CADDY

The caddy is of durable, flexible recyclable plastic like the funnel. As depicted here, the caddy is designed to clamp on both sides, east and west, of the commode. The caddy is contemplated to hold the standard urine specimen cup with or without the actual funnel as depicted in Drawing FIG. 1F.

The funnel is fitted to the caddy top. Though not depicted, the urine specimen cup is attachable to the bottom of the funnel as depicted in FIG. 1D.

DRAWING FIG. 1E URINE FUNNEL INVERTED/DETACHABLE FLIPSKIRT

The funnel is the same as FIGS. 1A-1C. The bottom is the same as the septa cap in FIG. 1A. The entire bottom is made of the flexible septa and is called a flipskirt because it can be flipped down and attached to the urine specimen cup as shown and can be perforated along the seam for detachment between the funnel and the septa flipskirt cup.

DRAWING FIG. 1F URINE FUNNEL WITH THE TOILET CADDY OPTION 2

This toilet caddy is one and the same as FIG. 1D. The funnel is the same as FIGS. 1A-1C, 1E-1F. The caddy fits the commode on east to west sides. The funnel basecap is not on the funnel and the specimen cup fits the funnel from beneath the caddy.

Drawings FIGS. 1G-1 H entail the same funnel depicted in FIGS. 1A-1C, 1E-1F as well as two stacked finger grooves or loops (originally shown in drawing 44 of Feb. 3, 2014 61/965,554 conf. 5746 application and included by handwriting) included beneath the narrower spout for stability in pouring from the upper funnel resevoir to other containers such as specimen cups.

The purpose of the invention is to:

1. provide an alternative mechanism for urine specimen collection at the doctor, hospital, oncologist, urologist, gynecologist, employer, military, law enforcement or rehab;

2. provide a sanitary, recyclable, reusable method of using the restroom when confronted with unsanitary or hazardous conditions, such as:
a. overcrowded public restrooms
b. rest stop areas and gas stations
c. woods and roadsides in emergencies or camping
d. porta potties at concerts and sports events
e. recreational facilities
f. international travel where there are no commodes or the accomodations lack seats
g. public transportation such as buses, trains, flights

3. A TOY for use in the sand at the beach or in sand boxes for making castles, forts or for use with clay or play dough in designing school volcanic projects;

4. An alternative emergency gas or fluid apparatus for insertion in the automobile gas tank;

5. A method to pour liquids into other containers.

With regard to uses 1-2, the individual using the same will no longer have to sit or squat to alleviate him/her/self. This will avoid unsightly stains on clothing and shoes from inevitably wet floors and toilet seats that typically accompany the use of old fashioned specimen cups without this apparatus.

I claim that this patent protects:

1. the function as a funnel for medical, legal and rehab use with and without specimen cups.

2. the function of the funnel for outdoor, travel, sporting and recreational use for play, arts and educational purposes as set forth herein above

3. use of the funnel for pouring other liquids or powders into containers.
